(19) United States
(12) Patent Application Publication
(10) Pub. No.: US 2005/0177088 A1
(43) Pub. Date: Aug. 11, 2005

(54) Mobile patient care system, particularly mobile heart-lung machine

(76) Inventors: Erwin Knott, Poing (DE); Andreas Hahn, Berg (DE)

Correspondence Address: SCULLY, SCOTT, MURPHY & PRESSER;400 GARDEN CITY PLAZA, GARDEN CITY, NY, 11530, US

(21) Appl. No.: 10/514,032

(22) PCT Filed: May 7, 2003
(86) PCT No.: PCT/EP03/04794

(30) Foreign Application Priority Data
May 7, 2002 (DE) 10220381.4

(51) Int. Cl. A61M 37/00

(52) U.S. Cl. 604/6.14

(57) ABSTRACT

Disclosed is a mobile patient care system comprising a patient care device (2, 5), a control unit (3), a power supply unit (4), and a support (6) to which the patient care device, the control unit, and the power supply unit are fixed and which can be suspended on the patient's bed by means of a holding device (8).

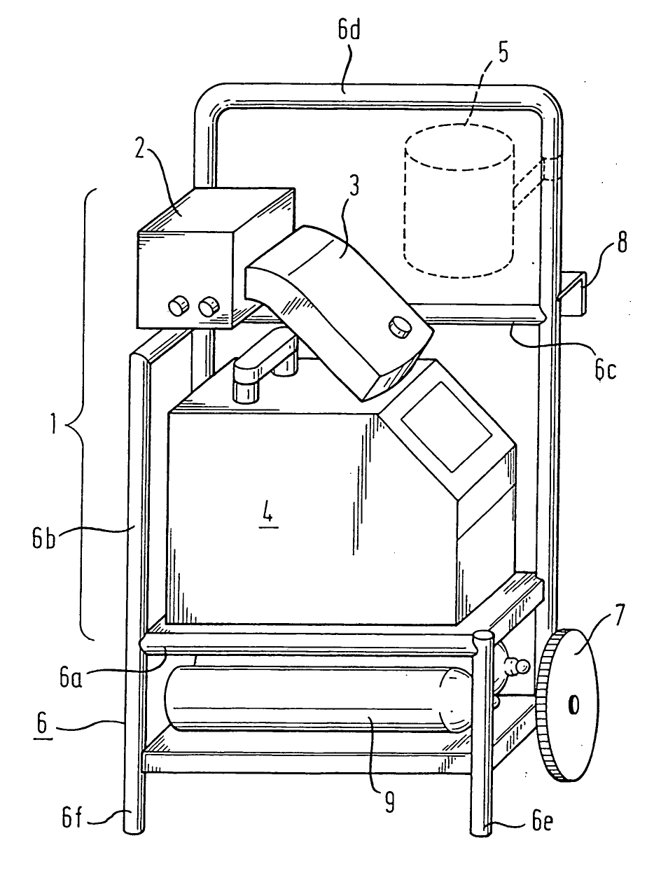

This invention relates to mobile patient care systems, such as mobile cardiovascular support systems, in particular mobile heart-lung machines (HLM).

The problem on which this invention is based is explained below on the basis of heart-lung machines, but it is also used with other patient care systems.

All the heart-lung machines (HLM) used in the hospital area are usually mobile, namely designed to be portable, so that the heart-lung machines can be transported out of a readiness room to the desired operating room and back again. Therefore, traditional heart-lung machines have several castors or wheels to allow the heart-lung machine, which is usually very heavy, to be moved. The great weight is attributed to the fact that the design of HLM for use in the hospital area is based on optimum care for the patient. Because of the weight of the HLM, the wheels or castors are designed to be load bearing and large accordingly.

In addition, there are emergency HLM systems for use in ambulance service, such as those known from U.S. Pat. No. 4,540,399. These systems are designed in an extreme manner so that the emergency workers are able to carry the emergency HLM by hand to the patient and to carry it beside the patient while transporting the patient. In addition, the limited space available in ambulances limits the size of emergency HLM systems designed for this intended application.

Such emergency HLM systems are not regularly used in the hospital because in the context of the facilities available in a hospital, these systems do not offer the reliable functioning and efficiency that is accepted in a hospital environment. On the other hand, it is also necessary in a hospital for patients connected to an HLM to be transported within the hospital. The essentially mobile (because portable) heart-lung machines for use in surgery are fundamentally suitable for being transported beside a patient bed, but because of the size and weight of both the patient bed with the patient and the heart-lung machine, reliable and safe transport can be achieved only with great effort. Several people are always necessary to move the patient bed and the HLM to which the patient is connected during the move, usually with the shortest possible tubing lines, in a coordinated manner. Great lengths of tubing are accepted of necessity because the HLM must always be moved again in relation to the patient bed. Frequently a project of this type will fail due to the size of doors or elevators or other building features. In other words, even in a hospital, there is a demand for a mobile HLM system which represents a balanced compromise between functional reliability, efficiency and mobility against the background of the hospital environment and in whose design building factors which are encountered repeatedly are also taken into account at the same time.

The goal of the present invention is to provide such a mobile patient care system, in particular a mobile heart-lung machine.

An inventive patient care system is described in Patent claim 1. Advantageous embodiments are derived from the subclaims.

An exemplary embodiment of an inventive patient care system is described in greater detail below on the basis of an inventive heart-lung machine with reference to the figure, which shows a perspective view of an exemplary embodiment of an inventive heart-lung machine.

The patient care device 1 as an exemplary embodiment of the inventive mobile patient care system illustrated in the figure is a heart-lung machine which also comprises, in addition to a pump device 2 and a control unit 3, a power supply unit 4 for supplying power to the pump device 2 as well as the control unit 3. The heart-lung machine also includes an oxygen enrichment device 5, a so-called oxygenator, for enriching the blood in the extracorporeal circulation of the heart-lung machine. During use, tubing lines (not shown here) are attached to the heart-lung machine to carry the patient's blood to the heart-lung machine and back to the patient. The pump unit 2 ensures that the blood is conveyed in the extracorporeal circulation, while the oxygenator 5 ensures that it is enriched with oxygen. The transport of blood in the extracorporeal circulation is controlled with the help of the control unit 3, which is also connected to the other components and allows the user to adjust at least the essential parameters of the extracorporeal circulation. Furthermore, the heart-lung machine illustrated in this exemplary embodiment includes a power supply unit 4, which ensures that both the pump unit 2 and the control unit 3 as well as other components of the heart-lung machine that rely on electric power, can be supplied with power to an adequate extent.

According to this invention, all the components of the patient care system, i.e., the heart-lung machine in this exemplary embodiment, are reliably mounted and secured on a carrying device 6. Thus an object that can be handled uniformly is created from the components of the patient care device and the carrying device 6 because all the components can be moved together because they are mutually secured together.

As shown in the figure, the carrying device 6 may be implemented in the form of a carrying frame of several straight and coiled pipe elements. The size and arrangement of the pipe elements, e.g., 6a, 6b, 6c, are such that the components of the patient care system are accommodated in the area encompassed by the carrying frame and are secured to the pipe elements and aligned with one another from the standpoint of operation.

The carrying frame 6, i.e., the carrying device, may easily be expanded by attaching additional pipe elements, e.g., by adding a handle 6d, which allows the user to move the inventive mobile patient care system. To do so, the carrying frame is preferably equipped with wheels 7 and/or castors 7, which make it possible for the inventive mobile patient care system to be moved like a trolley. To be able to stop the inventive mobile patient care system, the carrying device 6 has supporting elements 6e and 6f, the position and shape of which are such that together with the castors 7 and/or wheels 7 of the carrying device 6, they ensure secure standing. To move the mobile patient care system according to this invention, the user must tilt it and move it.

According to the essential aspect of this invention, the carrying device has at least one holding device 8 with which the carrying device 6 can be suspended from a patient bed, e.g., at the head or foot of the bed. The position of the holding device 8 is such that the carrying device 6 suspended from the patient bed is no longer in contact with the floor, so that the mobile patient care system can be moved together with the portable patient bed. The patient bed and the mobile patient care system according to this invention form a coupled unit, so this unit can be moved jointly in a very simple manner. By minimizing the number of components of the patient care device 1, the unit of the patient bed and mobile patient care system is only insignificantly larger than the patient bed itself. This means that in addition to the simple mobility of the unit as a whole, problems with respect to standard structural factors in a hospital are avoided because hallways, elevators, stairwells, basements, etc. are usually large enough to allow the patient bed, which has been increased insignificantly in size, to accommodate it or to allow it to pass by.

In an advantageous embodiment, a gas supply device 9 may be provided on the carrying device 6 of the inventive mobile patient care system, in particular for supplying the patient with oxygen and, in the case of a heart-lung machine, supplying the oxygenator of the HLM with oxygen.

In the simplest embodiment, the holding device 8 of the carrying device 6 includes at least one, but preferably two hooks 8 with which the carrying device 6 can be suspended from the foot or head part of the patient bed. The design of the hook 8 is preferably such that the carrying device may also be suspended from a rail system in the patient's room.

This invention convincingly makes use of the functionality of the wheels and/or castors of the bed provided by the portable bed on the one hand while on the other hand making use of the carrying capacity of the patient bed, which is not generally fully utilized. This ensures independent mobility through the handle 6d and/or the wheels 7 and supports 6e, 6f of the carrying device 6, which may be less pronounced in design because the movement functionality of the patient bed is utilized for the transport of the inventive mobile patient care system, which is relevant according to this invention, during the transfer of a patient, and a single mobile unit is thereby created, consisting of the patient bed and the patient care system.

Unlike portable patient care systems whose size is usually limited by the carrying capacity of a person, by utilizing the patient bed the patient care system can be moved with a greater weight because it is no longer necessary to rely on the carrying strength of the people involved. Therefore, with the mobile patient care system according to this invention, the design may be shifted much more in the direction of functional reliability and efficiency because mobility is now achieved by the portable patient bed.

Moreover, the use of this invention in conjunction with mobile heart-lung machines is very interesting because when the carrying device 6 is suspended from the foot part of the patient bed, comparatively short lines of tubing are necessary from the patient to the HLM and/or from the HLM to the patient. This circumstance is ensured not only by positioning the device at the foot end of the patient bed but also by the mechanically uniform connection of the patient bed and the mobile heart-lung machine to the carrying device according to this invention.

1. A mobile patient care system
a. having a device (2, 5) for providing a care function for a patient lying in a portable bed;
b. having a control unit (3) for controlling operation of the patient care device (2, 5);
c. having a power supply unit (4) for providing power for operation of the patient care device (2, 5) and the control unit (3); and
d. having a carrying device (6),
i. to which the patient care device (2, 5), the control unit (3) and the power supply unit (4) are attached in such a way that they can be moved together with the carrying device (6), and
ii. which has at least one holding device (8) with which the carrying device (6) can be mounted on the patient bed in such a way that the patient bed can be moved together with the carrying device (6).

2. The mobile patient care system according to claim 1, characterized in that the patient care device is a heart-lung machine having a pump unit (2) for conveying a patient's blood in an extracorporeal circulation and having an oxygen enrichment device (5) for enriching the blood with oxygen.

3. The mobile patient care system according to claim 1 or 2, characterized in that the carrying device (6) is a carrying frame.

4. The patient care system according to claim 3, characterized in that the carrying frame (6) consists of several incorporated pipe elements (6a-6d).

5. The mobile patient care system according to any of claims 1 to 4, characterized in that wheels or castors (7) for transporting the mobile patient care system without the patient bed are provided on the carrying device (6).

6. The mobile patient care system according to any of claims 1 to 5, characterized in that an area of the carrying device (6) is designed as a handle (6d).

7. The mobile patient care system according to any of the preceding claims, characterized in that a gas supply device (9) is provided in particular for supplying the patient and the heart-lung machine with oxygen.

8. The mobile patient care system according to any of the preceding claims, characterized in that the holding device (8) is designed in the form of at least one mounting hook (8) by which the carrying device (6) can be suspended on a patient bed, preferably at the foot or head end.

9. The mobile patient care system according to any of the preceding claims, characterized in that the carrying device (6) is not wider than a patient bed.

10. The mobile patient care system according to any of the preceding claims, characterized in that the carrying device (6) suspended from the patient bed does not come in contact with the floor.
